(19) United States
(12) Patent Application Publication
(10) Pub. No.: US 2012/0220049 A1
(43) Pub. Date: Aug. 30, 2012

(54) ASSAY METHOD AND DEVICE

(75) Inventors: Michael Bunce, Flint (GB); Benjamin John Passey, Liverpool (GB); David Charvill, Liverpool (GB)

(73) Assignee: Biofortuna LTD, Wirral (GB)

(21) Appl. No.: 13/262,623

(22) PCT Filed: Mar. 31, 2010
(86) PCT No.: PCT/GB2010/050573

§ 371 Date: May 15, 2012

(30) Foreign Application Priority Data
Mar. 31, 2009 (GB) 0905519.5

(51) Int. Cl. G01N 33/53

(52) U.S. Cl. 436/501; 422/69

(57) ABSTRACT

The present invention relates to a method and a device for detecting the presence of an analyte. More particularly, the present invention relates to the method and device for detecting the presence of immunoglobulins directed at polymorphic alloantigens such as HLA antigens and/or other products of the major histocompatibility complex (MHC).

The present invention relates to a method and a device for detecting the presence of an analyte. More particularly, the present invention relates to the method and device for detecting the presence of immunoglobulins directed at polymorphic alloantigens such as HLA antigens and/or other products of the major histocompatibility complex (MHC).

Transplantation and/or transfusion procedures often require that the subject should be screened for the presence of antibodies that might bind to donor antigens present on the donor tissue or blood; an example of these donor antigens being HLA antigens.

Methods known in the art for HLA testing include the complement-dependent lymphocytotoxicity (CDC) test in which serum from a recipient is incubated with donor lymphocytes followed by incubation with complement. The level of cytotoxicity is then estimated by discriminating between dead and viable cells using a dye. This method is labour intensive, requires viable cells, may be non-specific and requires a subjective evaluation.

Immunoassays, for example, utilize mechanisms of the immune systems, where antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a biological sample.

Enzyme linked immunoabsobant assays (ELISA) have been described for detecting anti-HLA antibodies (Zaer et al., Transplantation 63: 48-51 (1997); U.S. Pat. No. 6,046,013 & U.S. Pat. No. 5,482,841) whereby HLA class I molecules purified from pooled platelets or lymphoblastoid cell lines are immobilized in the wells of an assay plate and can be used to detect anti-HLA antibodies whereby the readout of the system is a change in optical density in the wells of the test plate. Barnardo et al (Transplantation. 2000 Aug. 15; 70(3):531-6) showed that single recombinant HLA rather than antigen from pooled cells or platelets could be used in an ELISA format for detecting HLA antigens.

More recently, Jar-How teaches in U.S. Pat. No. 5,948,627 that HLA antigens can be attached to microbeads and detected in a fluidic ELISA assay utilising a flow cytometer, Luminex platform or similar device for detecting bound anti HLA antibodies.

These antibody detection methods are slow and require expensive specialist laboratory equipment such as optical density plate readers or flow cytometric devices. The lack of a rapid point of care test means that all samples for alloantibody screening have to be stored and batch tested resulting in delays in the use of labile clinical blood, blood products and organs for transplantation.

Lateral flow tests or Immunochromuatographic assays, have been around for some time. They are a logical extension of the technology used in latex agglutination tests, the first of which was developed in 1956 by Singer and Plotz. The benefits of lateral flow tests include; rapidity of test (less than 10 minutes being typical), easy to use (typically just dip in sample), long-term stability making it suitable for worldwide use.

In their simplest form a lateral flow tests is a bibulous strip where a sample flows along the strip via capillary action drawn by an absorbent pad at one end of the bibulous strip. As the sample traverses the strip it encounters a coloured reagent, typically microparticles to which a specific binding member (typically a protein or antibody) has been conjugated. The coated microparticle mixes with the sample and binds its ligand. The bead-analyte complex transits the strip encountering lines or zones which have been pre-treated with an antibody or antigen. Depending upon the analytes present in the sample the coloured reagent can become bound at the test line or zone. Lateral Flow Tests can operate as either competitive or sandwich assays. Two types of chromatographic immunoassays are commonly described. In the one, proteins or small molecule analytes contained in human fluids (urine, blood, plasma, serum, and saliva) are detected. The analytes include hCG, FSH, LH, CKMB, TSH, troponins, myoglobulin, cancer proteins, viral/bacterial proteins, haptens, therapeutic drugs, and drugs of abuse.

In the other chromatographic immunoassay, the analyte being detected is human antibody specifically reactive with agents such as viral/bacterial proteins (HIV, Hepatitis A and C, H. pylori, EBV, Rubella, CMV, HSV, Dengue fever, Lyme, Chagas, TB, Toxoplasma, autoimmune antigens, etc.) or allergens (pollens, molds, dust/mites, foods, animal epithelia, etc.).

For detection of antibody in lateral flow tests various formats can be employed; the two common methods are competitive or non competitive. The competitive format relies on the analyte being tested being prevented from binding at the test zone on the strip by a labelled competitive agent; a negative test for the analyte results from a colour change at the test zone caused by the labelled competitive reagent. In competitive tests an irrelevant label is detected further along the test zone to show that the assay has worked. In competitive assays the test zone may be comprised of antigen or antibody; if antigen is used the competitive labelled reagent is anti-antigen which competes for binding at the test zone with test antibody. If antibody is used at the test zone in a competitive assay the competitive agent is a labelled antigen which is allowed to bind to its ligand in the test sample and thus prevents it binding at the test zone. Competitive assays are not as popular as non-competitive assays due to the fact that a negative visual result is actually a positive result for the test and thus seems counterintuitive, especially to the layperson.

In non-competitive assays a positive test zone is a positive test for the analyte: In these non-competitive tests the test zone may be the analyte or an antibody to the analyte. If the test zone is the analyte (the antigen) the mobile labelled reagent is typically anti-immunoglobulin. In this test the anti-immunoglobulin in the mobile phase labels all of the immunoglobulin in a sample, the labelled immunoglobulin traverses the strip and is captured at the test zone if antibody to the antigen is present resulting in a colour change at the test zone. If anti-immunoglobulin is used at the test zone the labelled mobile phase is the test analyte, which binds antibodies in the sample, traverses the strip and is captured by the anti-immunoglobulin at the test zone.

Lateral flow assays are a useful addition to laboratory tests as they are potentially rapid and economical methods of determining presence of a clinical analyte. However, existing devices are not without their problems and existing designs are not appropriate for all analytes. To improve lateral flow assay devices improved one-step assay devices and methods have been described. For example, the May et al U.S. Pat. Nos. 5,602,040, 5,622,871, 5,656,503, 6,187,598 and 6,228,660 disclose devices, kits and methods which facilitate one-step lateral flow assay methods. A test strip is provided with a dried labelled reagent which is released into a mobile form by a liquid biological sample. The labelled reagent specifically binds with the analyte to be detected to form a complex, and the migration of the liquid sample along the lateral flow matrix conveys the complex by capillary action to a detection zone.

Unrelated blood components can cause interference in lateral flow assays. Padhia et al in patent No WO/2007/063423 disclose a two-step method for the detection of IgE whereby the housing of the device contains filters for the removal of whole blood components such as cellular material which allows the use of the device in whole blood.

More specifically, a problem in lateral flow tests is the large excess of IgG in samples that may interfere with the detection of other classes such as IgE. To address this, Hubscher et al in U.S. Pat. No. 6,528,325 disclose a more specific device and method for detection of IgE antibodies in human serum by use of a lateral flow assay which facilitates one step techniques.

A test sample obtained from bodily fluids reacts with a gold labelled antigen and the resulting complex travels across a membrane and along a lateral flow strip. Coloured lines formed in specific locations along the test strip indicate the presence of class specific antibodies in the test specimen. In a more specific embodiment disclosed by Hubscher et al, the lateral flow assay has an IgG reacting protein (e.g. protein A or an antibody to IgG) added to the sample pad in order to complex the IgG contained in the sample such that the molecular weight of the IgG complex is greater than 1.0 million. This large complex travels sufficiently slower than IgA, IgM, and IgE thereby allowing these antibodies to react prior to the IgG. Accordingly this invention would not work for the detection of IgG as the IgG mobility is retarded leading to unbound IgG blocking the capture of specific bound IgG.

Accordingly, there is a requirement for improved methods of detecting antibody to alloantigen including methods for determination antibody which is rapid, convenient and accurate.

It is an object of the present invention to overcome or alleviate one or more of the problems associated with the prior art.

None of the prior art teach or disclose a fast and effective lateral flow method suitable for the rapid and sensitive testing of antibodies to polymorphic antigens such as MHC or HLA antigens or platelet glycoproteins.

In accordance with a first aspect of the present invention, there is provided a flow-through assay device for detecting the presence or quantity of an analyte residing in a test sample the device comprising a porous membrane in liquid communication with a conjugate pad and a wicking pad the conjugate pad having detection means for detecting a specific analyte, the porous membrane having a detection zone where an immobilized first capture reagent configured to bind to at least a portion of the analyte and analyte-conjugate complexes to generate a detection signal.

The present invention provides an improved, rapid method for detecting antibodies that allows the user to quickly evaluate presence or absence of antibodies in a biological sample to, e.g., HLA antigens, and in other embodiments to also determine the specificity of the antibody. The test may be a clinical point-of-care (POC) test, and/or a home based POC test based on finger-prick whereby a patient can check antibody status at home. The test comprises the step of adding biological fluids from a subject to a lateral flow device: Biological fluids containing relevant antibodies flow along the device and are contacted by antigen ligand conjugated to coloured, fluorescent and/or gold micro particles. Detection of analyte-bound antibody occurs at the test line whereby the same or similar antigen is immobilised: the antibody-antigen-particle aggregate is immobilised by binding of the antibody to the antigen at both locations (bead and strip) and the positive test is indicated by the visual presence of the coloured beads. Throughout this application it should be clear that HLA antigens are used by example and are preferred embodiments of a polymorphic alloantigen series and that the invention comprises lateral flow tests for the detection of any alloantibodies to any alloantigens.

Preferably, a control zone is located downstream from the detection zone on the porous membrane and has a second capture reagent immobilized within the control zone.

In one embodiment, the conjugate pad is located upstream from the detection zone, and has detection probes with specific binding members for the analyte.

The detection means preferably comprises a visual marker such as a coloured particle, and/or directly attached chromophore and/or fluorophore for conjugating to and marking/labelling the ligand that allows detection of the analyte.

The conjugate pad contains detection means that signal the presence of the analyte. The conjugate pad may also include other, different detection means populations, including means for indication at the control zone. The detection means may be nanoparticles, e.g., in the diameter range of 5-150 nm, more particularly the means have a diameter between 20 and 60 nm, in order to facilitate the mobility of the marker-analyte conjugate. Suitable detection means include those made from metal colloids, such as silver and gold colloids, fluorescent particles, carbon, polystyrene and dyed latex particles.

In one embodiment, a sample containing an analyte is deposited on the conjugate pad, interacts with the detection means, and moves toward the detection zone for detection.

The terms “upstream” and “downstream” refer to the position of an item relative to the direction of flow of a sample from the point of deposition on the assay device.

The term “analyte” generally refers to a substance to be detected. For instance, analytes may include polymorphic antigens such as MHC or HLA antibodies or platelet glycoproteins, haptens, antibodies, and combinations thereof. Analytes include, but are not limited to, toxins, organic compounds, proteins, peptides, microorganisms, amino acids, nucleic acids, hormones, steroids, vitamins, drugs, drug intermediaries or byproducts, bacteria, virus particles and metabolites of or antigens to or immunoglobulins directed to any of the above substances.

The term “test sample” generally refers to a material suspected of containing the analyte.

The term “liquid communication” refers to the ability of a liquid and its contents, including analytes etc., to be capable of moving from one zone to another.

The test sample may, for example, include materials obtained directly from a source, as well as materials pre-treated using techniques, such as, but not limited to, filtration, precipitation, dilution, lysing, distillation, mixing, concentration, inactivation of interfering components, the addition of reagents, and the like. The test sample may be derived from a biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, menses, semen, feces, amniotic fluid or the like.

The wicking pad is preferably in liquid communication with the membrane and provides a driving force for liquid movement due to the capillarity of the pad.

The present invention thus provides a test device detecting the presence of clinically relevant antibodies to polymorphic antigens involved in transplantation and blood products transfusion. The test device is readily usable by a trained technician or an untrained person and which preferably simply requires application of sample to the sample pad (e.g. blood from a finger prick or blood sample or serum sample) and thereafter few or no further actions are required by the user prior to the analytical result being observed. Ideally the analytical result should be observed rapidly, within ten minutes or less.

The present invention relates to rapid laboratory and point of care methods for detection of antibodies to transplantation or transfusion alloantigens. Alloantigens are antigens existing in alternative (allelic) forms in a species, thus inducing an immune response when one form is transferred to members of the species who lack it; examples of these include anti-human leukocyte antigen (HLA) antibodies and blood group antibodies directed against human platelet antigens (HPA) and erythrocyte antibodies. Individuals may be sensitized to alloantigens during pregnancy, or by blood transfusion or platelet transfusion or previous organ grafts. Exposure to HLA alloantigens can generate an antibody response that may be of any of the immunoglobulin classes, but is typically IgG, IgM, or rarely, IgA. Detection of alloantibodies in an individual is important in many clinical situations such as transfusion and transplantation. Testing to determine sensitivity to HLA alleles is relevant to tissue and organ transplantation where the presence in the recipient of antibodies against HLA antigens of the donor is predictive of a high risk of graft rejection. Testing for anti-HLA antibody post-transplant can also be of clinical relevance as the appearance or increase in anti-HLA antibodies may correlate with graft rejection. Detecting IgG class of immunoglobulin is of prime importance as this class is the most deleterious to transplant success, and consequently most antibody detection systems rely on IgG detection. It is a standard practice in the transplant field to test all potential recipients against a panel of HLA antigens selected to represent a human population and the percentage of HLA alleles against which the serum is reactive is determined. This process can be lengthy and costly and in some circumstance it is important that a very quick result is obtained. Time and money can be wasted in conventional testing if the sample does not have HLA antibodies, thus there exists a need for a quick and cheap screening test that identifies the presence of deleterious antibodies. Further, the presence of antibodies to polymorphic antigens such as HLA within blood or blood products such as whole blood or platelet transfusions can cause transfusion-related acute lung injury (TRALI) in the recipient if the transfusion contains anti-HLA antibodies; accordingly the invention is eminently suitable to the screening and identification of deleterious antibodies in blood products.

The device may be a simple dipstick whereby the sample pad is inserted into the serum sample or blood sample. Alternatively it may further comprise a housing with apertures above the sample pad to allow the addition of sample and windows at the test zones for viewing the result. Alternatively, multiple assays can be incorporated into a single serum or blood collection device as demonstrated by multiple analyte urine analysis devices such as urine cups that detect drugs of abuse. In this last embodiment it would be expected that a device would house between 1 and 50 test strips, each test strip having between 1 and 50 alloantigen zones allowing for up to 2,500 single alloantigens to be tested in one go. The device receptacle would accept a volume of reagent to analyse and would allow a certain amount to be applied to each individual test simultaneously.

In another embodiment the test device can be used integrally with blood product collection bags to give a visual indicator of the alloantibody status of the blood product.

In accordance with a further aspect of the present invention, there is provided a method for detecting the presence or quantity of an analyte residing in a test sample comprising:

i) providing a flow-through assay device comprising a porous membrane in liquid communication with conjugate pad and a wicking pad, the porous membrane defining:

a) a detection zone within which a first antibody is immobilized that is configured to bind to complexes formed between the analyte and the conjugated detection means to produce a detection signal;

b) a control zone within which is immobilized a second antibody, capable of producing a control signal when contained within the control zone;

ii) contacting a test sample containing the analyte with the conjugated detection means; and

iii) detecting the detection signal.

In accordance with a further aspect of the present invention, there is provided a kit of parts for detecting the presence or quantity of an analyte in a test sample comprising:

a) a device as described hereinabove;

b) wash buffer and/or chase buffer; and

c) detection means.

In accordance with a further aspect of the present invention, there is provided a method for detecting the presence or quantity of an analyte residing in a test sample comprising:

a) contacting an analyte containing sample with detection means;

b) removing unbound detection means and/or other materials by washing;

c) providing a flow through any device having a detection zone within a first antibody is immobilised that is configured to bind to complexes formed between the analyte and the conjugated detection means to produce a detection signal, and a control zone within which is immobilised a second antibody, capable of providing a control signal when contained within the control zone;

d) contacting a test sample containing the analyte within the conjugated detection means; and

e) detecting the detection signal.

A specific embodiment of the present invention will now be described, by way of example only, with reference to the accompanying figures, in which:

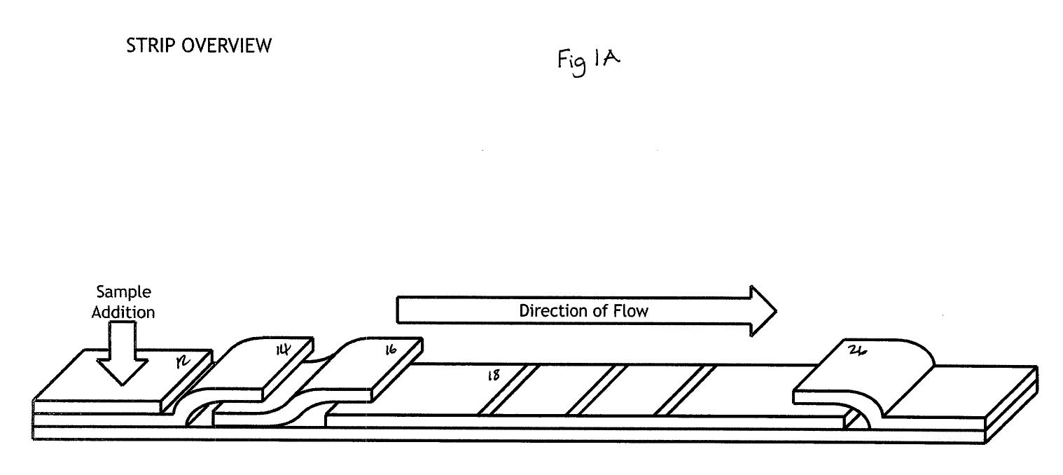

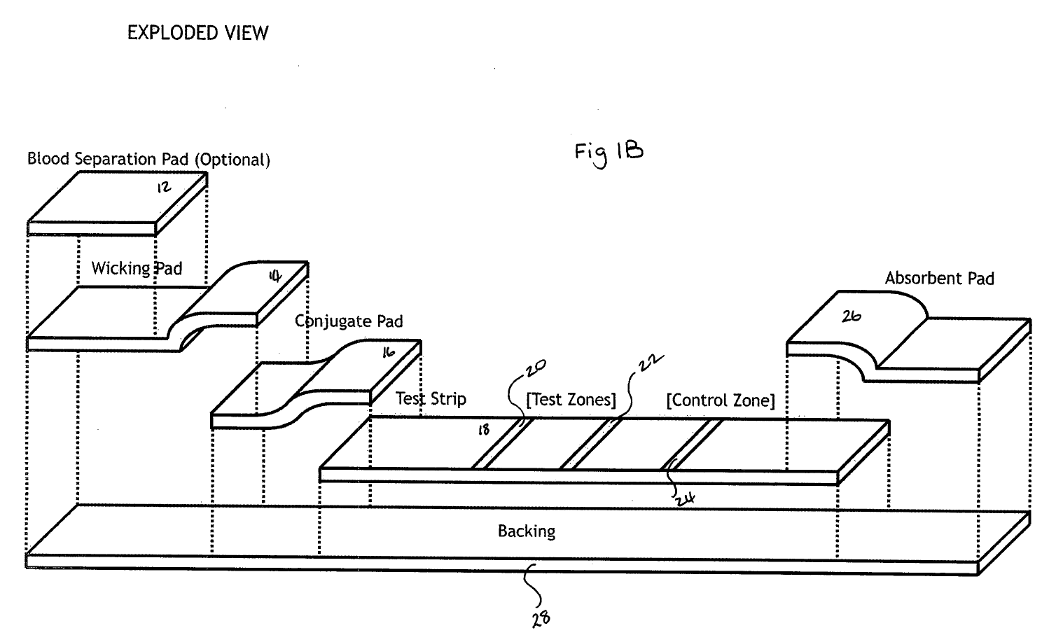

FIGS. 1a and b are schematic diagrams of a lateral flow assay device in accordance with the present invention;

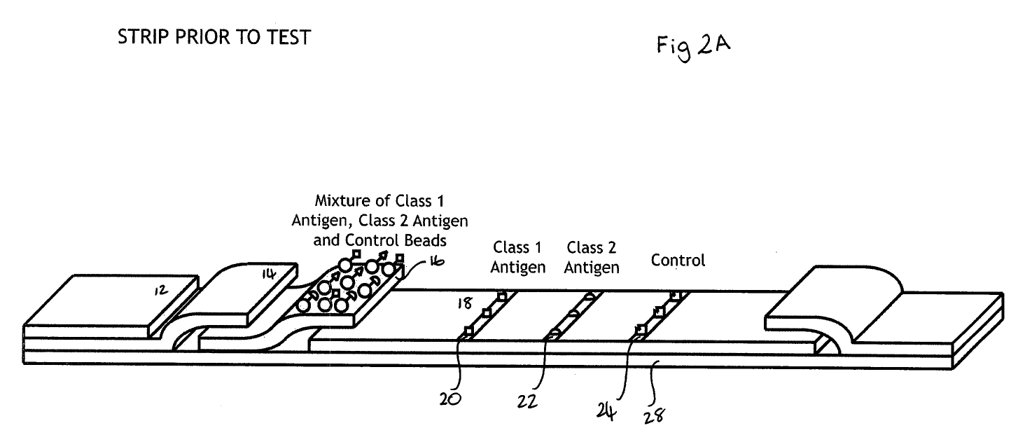

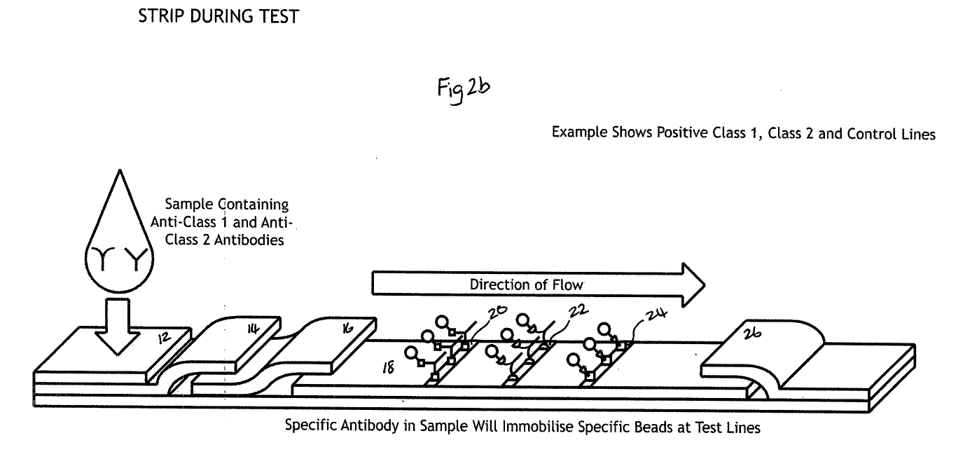

FIGS. 2a and b are schematic diagrams of the lateral flow assay device of FIGS. 1a and b;

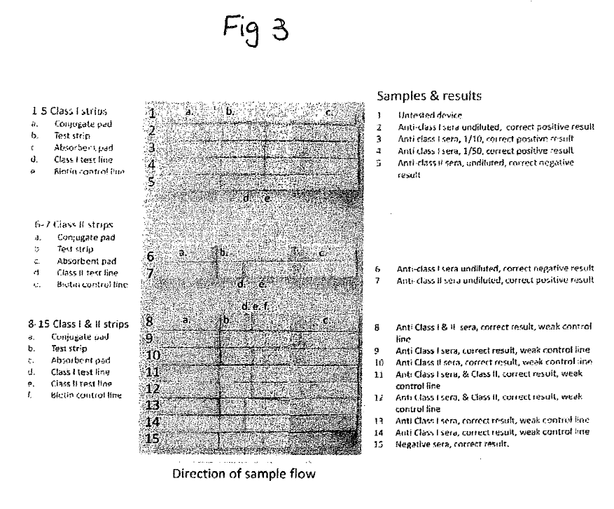

FIG. 3 shows dipstick tests in accordance with the present invention.

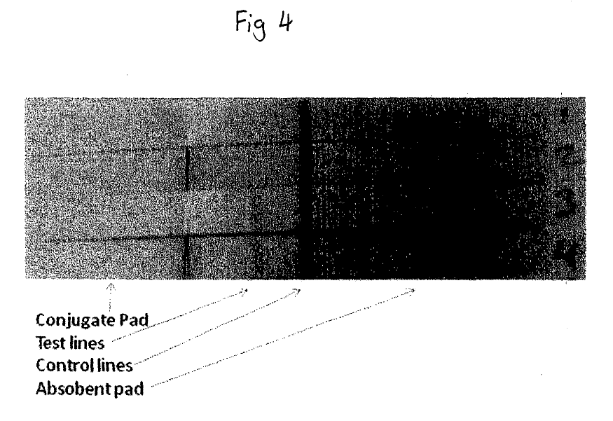

FIG. 4 shows the results of the two-stage assay of example 16 in accordance with the present invention; and

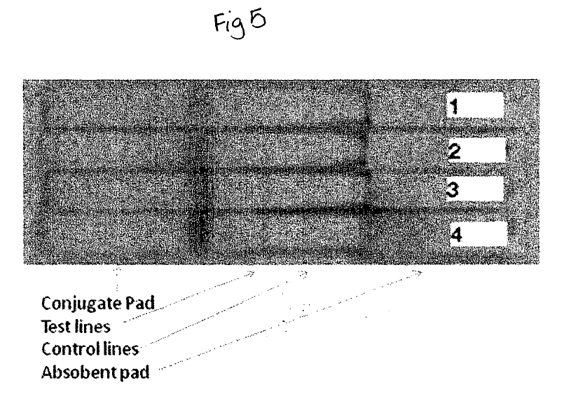

FIG. 5 shows the results of the two-stage assay of example 17 in accordance with the present invention.

The device 10 of the present invention minimally comprises a lateral flow device or lateral flow dipstick comprising the components shown in FIG. 1, namely an optional blood separation pad 12, a wicking pad 14, a conjugate pad 16, a backed test strip 18 containing immobilised antigens (test analytes) and control analytes, 20, 22, 24 divided in to test zones and control zones and an absorbent pad 26 mounted on a support 28. The test strip may be provided un-housed as a dipstick test whereby the sample pad is merely dipped into the analyte, or; the lateral flow test device may be contained in a plastic housing that obscures the strip and just reveals the sample pad, test and control zones; in this embodiment the sample is applied by dropping onto the exposed sample pad window. Alternatively, the lateral flow device may be housed with a plurality of lateral flow dipsticks specific for different analytes and housed in a general receptacle to allow a plurality of tests to be performed simultaneously for a single sample.

HLA antigens are used by example of a polymorphic series and that the invention pertains to lateral flow tests for the detection of any alloantibodies.

In the first embodiment (as shown in FIG. 2) the lateral flow test is used to simultaneously detect and discriminate between immunoglobulins directed at polymorphic HLA class I antigens and in another detection zone, class II antigens:

The test device consists of an optional blood filter membrane, a conjugate pad containing the mobile phase labelled reagents, a backed test strip made typically of nylon or nitrocellulose that allows the applied sample to migrate to test and control zones of immobilised ligands and an absorbent pad to soak up the spent sample and to encourage wicking along the strip. The first embodiment utilises a mobile phase where the test beads are polystyrene beads of a particular colour that are conjugated to affinity purified HLA class I antigens from a pool of donors; another colour bead, differentiated from the first bead colour, is conjugated to class II antigens. The pool of donors is selected so that the majority of common HLA antigens are represented in the pool. The same pool of antigens are coated at high concentrations at independent locations forming two test lines; a class I test zone and a class II test zone. The test beads are dried onto the conjugate pad alongside a control bead population, preferably utilising a third colour bead, coated with streptavidin. The control zone consists of biotin conjugated to bovine serum albumin (BSA). In a positive test the sample is applied to the sample pad. The use of a blood separation filter such as Vivid Plasma Separation membrane (Pall Ltd) allows the sample to be blood or serum or any other biological fluid potentially containing antibodies; the antibody containing component traverses the plasma separation membrane into the conjugate pad, whereby the immunoglobulins encounter and bind to the HLA antigen. The wicking effect of the test strip then draws the sample containing beads, immunoglobulin and control beads along the strip. As the antibody bound beads encounter the immobilised antigen the bead/immunoglobulin complex will become immobilised. This phase works on the basis that IgG and IgM immunoglobulins have two and ten valencies for antigen respectively. Thus in the cases of IgG binding one part of the IgG molecule binds the mobile phase and the other part of the molecule binds the immobile phase. In the event of a negative test no antigen coated beads are immobilised at the test zones, however the streptavidin coated beads are immobilised at the distal end of the test strip by the biotin ligand. This is utilised to show that all of the reagents have correctly traversed the test zones.

The first embodiment the test identifies the presence or absence of antibody to alloantigen pools: In a second embodiment various combinations of single or plural antigens can be used to identify the specificity of the antibody. For example, single antigens made by recombinant biology methods or single antigens or single donor antigens (the sum of one individual's antigens) cleaved from cell membranes and affinity purified can be applied in either the mobile phase, the test zones or in both. In this embodiment single antigens are applied in the immobilised zone, one alloantigen per zone, whilst the same single antigen, or a pool of the relevant class of antigen is conjugated to beads in the mobile zone. Thus immunoglobulin binds to the antigen in the mobile zone and is carried to the test zone; a positive result would indicate the presence of specific antibody to that single antigen. Multiple test zones can be used, each test zone containing a different single antigen, or antigens. Likewise, in a third embodiment peptide fragments could be used to identify the epitope.

In a fourth embodiment the unique feature of the test, i.e. sample immunoglobulin sandwiched between mobile antigen and fixed antigen allows for two different antigens to be used at the two locations and thus allows for the immunological phenomenon of cross-reactivity to be examined. In this test the mobile phase would feature antigen, e.g. A0101, the multiple immobilised test zones would feature the same antigen in one test zone (the most distal test zone) and related (crossreactive) antigens at other test zones, e.g. test zones 1,2 & 3 might contain A3601, A1101 and A0101 respectively. The basis of this test is that each arm of the divalent IgG molecule is identical but each individual arm can bind to similar epitopes on different alloantigens with varying affinity or avidity, known by those skilled in the art as cross-reactivity. In this test if an antibody is cross-reactive with A0101 and A3601 the divalent antibody will link the bead to the antigen in test zones 2 and 3. If an antibody was cross-reactive between A0101 and A1101 then the result would show positive reactions at zones 2 and 3. In this manner the specificity of alloantibodies can be dissected, the information from which can be of clinical value to those skilled in the art of monitoring patients' antibody response in transplant or transfusion situations.

As the test in the first to fourth embodiments does not discriminate between immunoglobulin classes (IgG, IgM etc) or isotypes (IgG1, IgG2 etc) a fifth embodiment can be used whereby an additional indicator can be used to indicate the class or isotype of antibody present. In this embodiment labelled class or isotype-specific antibody, e.g. anti-IgG is added in the conjugate pad, or afterwards in a chase buffer whereby the labelled antibody would specifically recognise the class or isotype of the bead-antibody-antigen complex present at the test zone. The labelled antibody would need to have a different label to any of the labelled antigens or controls in order to allow visual discrimination. This label may be a different coloured particle or a directly labelled fluorescent marker such as Cy3 or Cy5 or any other fluorophore that could be visualised by the appropriate excitation.

The lateral flow device can be constructed using the components as shown in FIG. 1. The optional blood separation layer acts is a filter that removes particulate matter, in particular clots, fibrin and cellular material from whole blood allowing the antibody containing fraction, the plasma, to enter the next phase, the wicking pad. The filter is usually constructed from permeable glass fibre impregnated with pyols that clot blood components preventing transmission of non-soluble components as described in U.S. Pat. No. 5,725,774, a typical example being Vivid™ Plasma separation membrane (Pall Ltd). In the absence of a blood separation filter lateral flow devices are not usually recommended for use with whole blood. The next stage is the sample pad that serves to transfer the analyte to the conjugate containing pad. The conjugate pad is usually constructed of glass fibre which allows the ligand-bead conjugate to be dried onto in such a way that when the motile analyte stage reaches it the beads are freed from the glass fibre solid support and the analyte, ligand-bead and liquid components transfer onto the bibulous test strip. The test strip serves to transfer the reactants and liquid from the conjugate pad area to the distal absorbent pad and allowing the components to contact fixed reagents dispersed in discrete zones on the strip (the test and control zones). The strip should be made of any material that allows the reagents to traverse the trip in a controlled manner that allows the reactant to contact the test and control zones. The test strip is typically made of a porous membrane constructed from Acrylic co-polymer, cellulose acetate, nitrocellulose, nylon, polyethersulfone (PES), polypropylene, polysulfone, polytetrafluoroethylene (PTFE), or polyvinylidene fluoride (PVDF). The test and control zones are typically sprayed and dried onto the membrane in 1 mm wide zones with the control always distal to the test zones to ensure a completed test. The distal absorbent pad can be made of any absorbent material suffice that it should be able to absorb the total liquid volume added. The assembled strip can be contained within a plastic housing as a typical lateral flow test or can be used without the housing as a ‘dipstick’ in which case the proximal end is inserted into the analyte fluid and allowed to wick along the dipstick to complete the test.

With reference to FIG. 3, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used but some experimental errors and deviations should be accounted for, unless indicated otherwise.

For working examples of the invention three different devices were constructed. The test membrane used for all tests is from Sartorius; UniSart CN140 constructed of cellulose nitrate polymers (laminated on the reverse side with impermeable backing material), part number 1UN14ER050025: device 1 contained test strips containing a single test zone consisting of a pool of HLA class I antigen, and a separate distal control zone consisting of biotin; the second device consists of a pool of HLA class II antigen, and a separate distal control zone consisting of biotin; the third device contains two test zones whereby class I and class II antigens are applied in two distinct test zones with a distal control zone of biotin. In each case the HLA antigens were purchased as affinity purified human MHC antigens from GTI Inc (Waukesha, Wis. 53186 USA) and both Class I and Class II antigen pools were applied at a concentration of 0.75 mg/ml at a rate so that the test device received approximately 0.5 ul per device. In each case protein was diluted from stock in a 0.01M borate buffer containing 5% (w/v) BSA and 20% (w/v) sucrose. Borate buffer is 0.8 g Sodium chloride, 381 g Borax (Sodium tetraborate decahydrate) and 1 litre of distilled water mixed, adjusted to pH8.5 and autoclaved prior to use. Biotin control zones were constructed by applying 2 mg/ml BSA-Biotin conjugate at a rate that delivers approximately 0.5 ul per device. After striping the test ligands and controls the membrane is air dried and then blocked by applying Stabilcoat™ from SurModics, Inc. (Eden Prairie, USA).

For the mobile phase of the test the same class I and class II antigen pools from GTI Inc are conjugated to Bangs Labs DC02B Dark Blue Polystyrene microspheres with —COOH active group and a mean diameter of 0.43 um. The protocol for this is as follows:

1. Mix 50 ul beads (10% concentrate) with 1450 ul wash buffer.

2. Centrifuge at 21000-21500 g for 4 minutes.

3. Wash beads & repeat centrifugation twice and re-suspend in wash buffer

4. Remove supernatant and re-suspend in 125 ul wash buffer (use 200 ul pipette if possible for this stage—by gentle washing action once again).

5. Incubate at 35° C. for two hours under continual rotation.

6. Add 125 ul blocking buffer.

7. Incubate for 30 minutes whilst rotating.

8. Centrifuge as previously for 4 minutes.

9. Remove supernatant and re-suspend in 750 ul wash.

10. Wash beads & repeat centrifugation twice and re-suspend in wash buffer, centrifuge again and remove supernatant and re-suspend in 250 ul storage buffer. Store at 4° C.

The ‘wash buffer’ is borate buffer ph 8.5.

The ‘blocking buffer’ is prepared using borate buffer with the inclusion of 4% BSA w/v.

The ‘storage buffer’ once again is based on the borate buffer with inclusion of 5% BSA and 20% sucrose w/v (+0.01% sodium azide as preservative).

The immobilised phase (the striped and blocked strips) was assembled as dipsticks: a glass fibre conjugate pad was attached to the proximal end and the absorbent pad to the distal end. The dipsticks were stored at ambient temperature until use.

Examples 1 to 15 are shown as dipstick tests (see FIG. 3), i.e. the test strip was not sealed in a standard plastic lateral flow housing, however it is to be understood that a similar process and procedure would be used in a finished item as goods for sale. It would be understood by those skilled in the art that the concentration of reagents used in the lateral flow manufacture, and the volumes of test sample used may vary in any clinically successful product.

### EXAMPLE 1

Example 1 is an untested lateral flow dipstick for comparison purposes.

For the examples 2-5 shown in FIG. 3, 1 ul of class I conjugated test beads and 0.5 ul of 1% solution 0.32 um streptavidin beads CP01B/8891 (Bangs Labs) were applied to the conjugate pad. Subsequently 40 ul of serum samples gifted by Oxford Tissue typing laboratory Oxford, and known to contain HLA antibodies were applied to the conjugate pad with a Gilson pipette. The samples were ‘chased’ with PBS buffer containing 1% v/v tween 20 by adding 40 ul of chase buffer to the well of a 96-well microtitre plate and placing the strip into the well so that the conjugate pad absorbed the chase buffer which allowed the sera, and bead-conjugate to travel vertically up the strip past the test and control lines and onto the absorbent pad. The test was completed within 10 minutes when the control line appeared.

### EXAMPLES 2-4

Examples 2-4 were the same Anti-B18 sera sample tested undiluted, 1/10 (diluted in PBS), and 1/50 dilution. All samples showed positive class I test line that weakens in strength as the sample is diluted.

### EXAMPLE 5

Example 5 was a sample positive for class II antibodies directed at DR2 and DQ1, this is thus shown correctly negative with the class I test line.

### EXAMPLES 6 AND 7

Examples 6 and 7 utilise a strip containing only class II striped at the test zone, and again biotin at the control zone. For the examples 6-7 shown in FIG. 3, 1 ul of class II conjugated test beads and 0.5 ul of 1% solution 0.32 um streptavidin beads were utilised as described previously. Sample 6 is a class I antisera, shown correctly negative against the class II line, whilst test 7 shows the successful identification of the class II antibody containing sera.

### EXAMPLES 8-15

These examples utilise a test strip that contains both class I and class II antibodies in different combinations. For the examples 8-15 shown in FIG. 3, 1 ul of class I and 1 ul of class II conjugated test beads were used with and 0.5 ul of 1% solution 0.32 um streptavidin beads. Presence of anti-class I is demonstrated by a line at the first position (d.), antibodies to class II are demonstrated by a positive line at the second position (e.). Some of the test sera are positive for the presence of both class I and II antibodies and therefore show two positive lines, conversely, sample 15 is a negative control sample that shows no class I or class II antibodies.

### Two-Stage Assay

Alternative forms of the lateral flow assay for HLA antibody include two stage assays where the analyte is contacted with the HLA ligand before being applied to the reactive areas of the lateral flow device. The advantage of this method is that it allows for the antigens to contact the sample antibody and irrelevant proteins or other materials in the sample can be removed before the bead-antigen-immunoglobulin complex traverses the lateral flow device. This methodology also allows for the assay to be used in alternative detection methods. Some examples of these are shown below.

### EXAMPLE 16

### Two Stage Assay Variation 1

To test the two stage assay Class 1 HLA proteins were conjugated to latex microparticles as per protocols described. The conjugated particles were mixed 1:1 with control biotinylated latex particles. 1 ul of the bead cocktail was added to the following positive and negative test samples.

1) 50 ul Anti-HLA class II (DRB) monoclonal (negative test)

2) 50 ul HLA class I negative sera (negative test)

3) 50 ul Anti-HLA A2 monoclonal antibody in 50 ul PBS Tween (positive test)

4) 50 ul sera positive with anti-class I HLA (positive test)

Each test was pelleted by centrifuging for 5 minutes at 14,300 rpm whereupon the supernatant was discarded. The latex particles were then resuspended in 50 ul PBS Tween and run on test lateral flow strips. The test line on these strips was diluted mouse anti-human IgG resulting in a positive control line for the positive samples. The test line was a streptavidin control line which bound the uncojugated beads showing the assay had completed. The results are shown in FIG. 4.

The test line in this two stage assay may be anti-IgG as in this case, or any other class or isotype specific example, such as anti IgM, anti IgG/A/M, anti siotype such as IgG1, 2, 3 or 4.

### EXAMPLE 17

### Two Stage Assay Variation 2; Divalent Antigen Test.

The same sera and monoclonals from example x were run on alternative strips that contained concentrated HLA protein on the test line instead of antibody, in a ‘divalent’ test as described earlier. The results are shown in FIG. 5.

Other variations of the one or two stage assays include a sandwich assay using a secondary antibody; in this example HLA coated beads are contacted to patient sample, if the sample contains an antibody to the HLA it binds. The bead-antibody complex then comes into contact and binds with anti-human antibody either in a separate buffer or in the conjugation pad. Examples of these secondary antibodies may be any mouse anti human IgG, goat anti human IgG or chicken anti human IgG. Any species of anti human immunoglobulin may be utilised, and again these can be class or isotype specific antibodies. To detect the bead-HLA-antibody-second layer complex an anti-species antibody is used at the test line; so if chicken anti human is used as a second layer the test line would consist of a suitable anti-chicken antibody that would immobilise the complex generating a positive response.

1. A flow-through assay device for detecting the presence or quantity of an analyte residing in a test sample the device comprising a porous membrane in liquid communication with a conjugate pad and a sample pad,
the conjugate pad having a first capture reagent for conjugating to a specific analyte to form an analyte-conjugate,
the porous membrane having one or more detection zones where an immobilized second capture reagent configured to bind to at least a portion of the analyte-conjugate complexes to said analyte-conjugate to generate a detection signal.

2. A device as claimed in claim 1, wherein the analyte comprises HLA antigens, polymorphic proteins or fragments of proteins that might elicit an antibody response in a transplant or transfusion situation, Minor histocompatibility antigens (MIHAg), granulocyte antigens (HNA series), platelet thrombocyte antigens (HPA series), Endothelial-monocyte antigens, CD31, or blood transfusion antigens, or immunoglobulins directed thereto.

3. A device as claimed in claim 1, wherein the test can be used to identify presence or absence of immunoglobulin directed at a polymorphic analyte.

4. A device as claimed in claim 3, wherein the polymorph is analyte comprises a mixed pool of antigens comprising many phenotypes or immunoglobulins directed thereto.

5. A device as claimed in claim 1, wherein the device is configured to identify
the presence or absence of immunoglobulin directed at a single analyte from a polymorphic system or an immunoglobulin directed thereto.

6. A device as claimed in claim 5, wherein the analyte comprises a single HLA antigen or a peptide fragment of a single antigen representing a clinically relevant epitope.

7. A device as claimed in claim 1, wherein the analyte comprises antigens derived from a plurality of sources combined to generate a pool of protein representative of all polymorphic antigens at a locus, or antigens derived from a single individual.

8. (canceled)

9. A device as claimed in claim 6, wherein the analyte comprises HLA antigens, and the device comprises a first detection zone that detects HLA class I antigens and a second detection zone that detects HLA class II antigens to allow multiple immunoglobulin specificities to be detected within an individual.

10. A device as claimed in claim 1, wherein at least one detection zone contains a ligand that is the same as a ligand as the mobile phase.

11. A device as claimed in claim 1, wherein at least one detection zone contains an antigen or epitope to an antigen or epitope in the mobile phase that allows for immunological cross-reactivity to be detected by linking an epitope of a first antigen to an epitope on a second antigen whereby the epitopes may be similar or different.

12. A device as claimed in claim 1, wherein the mobile phase consists of mobile particles composed of latex, polystyrene, polycarbonate, colloidal metal that can be detected.

13. A device as claimed in claim 1, wherein the sample pad is composed of a material that allows separation of serum from whole blood and allows for the flow of serum onto said device.

14. A device as claimed in claim 1, wherein the mobile phase comprises a plurality of differentially labeled particles that can be discriminated at different detection zones.

15. A device as claimed in claim 14, wherein the labeled particles comprise HLA Class I antigen, HLA Class II antigen and/or a control antigen.

16. A device as claimed in claim 15, comprising HLA class I attached to red particles, HLA class II attached to blue particles and control antigen attached to black particles.

17. A device as claimed in claim 14, wherein said mobile phase further comprises additional labeled antibodies directed to immunoglobulins whereby discrimination of antibody classes or isotypes is achieved.

18. A device as claimed in claim 1, wherein the correct flow of the device is tested by using an irrelevant label on particles in the mobile phase and a capture reagent for said label at a distal detection zone.

19. A device as claimed in claim 1, configured for use in pre or post-transplant monitoring and screening of blood and other blood products for the presence of antibodies and where said device is unhoused as a dipstick test whereby the sample pad is dipped into the analyte.

20. A device as claimed in claim 1, which further comprises a housing and an absorbent pad.

21. A device as claimed in claim 1, housed with a plurality of other devices specific for different analytes and housed in a general receptacle to allow a plurality of tests to be performed simultaneously for a single sample.

22. A device as claimed in claim 1, wherein the result can be discerned by eye or by a device that measures the intensity of the test and control zones to give a digital output that yields quantitative or semi-quantitative results.

23. A method for detecting the presence or quantity of an analyte residing in a test sample comprising:
contacting a test sample containing the analyte with the sample pad of the device of claim 1; and
detecting the detection signal.

24. A kit for detecting the presence or quantity of an analyte in a test sample comprising:
a) a device as claimed in claim 1;
b) wash buffer and/or chase buffer; and
c) detection means.

25. A kit as claimed in claim 24 further comprising a vessel for collecting a sample and which contains said detection means.

26. A method for detecting the presence or quantity of an analyte residing in a test sample comprising:
a) contacting a test sample containing the analyte with detection means;
b) removing unbound detection means and/or other materials by washing;
c) providing a flow through assay device having (i) a detection zone comprising an immobilised a first antibody configured to bind to complexes formed between the analyte and the conjugated detection means to produce a detection signal, and (ii) a control zone within which is immobilised a second antibody, which provides a control signal when contained within the control zone;
d) contacting the washed analyte conjugated to the detection means with the device; and
e) detecting the detection signal.

27. A two stage assay for detecting the presence or quantity of an analyte in a test sample comprising:
a first stage comprising:
a) contacting a test sample containing the analyte with detection means;
b) removing unbound detection means and/or other materials by washing;
and a second stage comprising:
c) providing a flow through assay device having (i) a detection zone within a first antibody is immobilised that is configured to bind to complexes formed between the analyte and the conjugated detection means to produce a detection signal, and (ii) a control zone within which is immobilised a second antibody, capable of providing a control signal when contained within the control zone; d) contacting the washed analyte conjugated to the detection means with the device; and e) detecting the detection signal.
